SEQUENCE LISTING (27 sequences)

SEQ ID NO: 1 - DNA, 15 bp, artificial sequences
  source 1..15: /mol_type=“DNA” /note=“Anti-mir” /organism=“artificial sequences”
attcacaacaccagc

SEQ ID NO: 2-10 - skipped (9 entries)

SEQ ID NO: 11 - DNA, 17 bp, artificial sequences
  source 1..17: /mol_type=“DNA” /note=“Antimir” /organism=“artificial sequences”
gattcacaacaccagct

SEQ ID NO: 12 - DNA, 20 bp, artificial sequences
  source 1..20: /mol_type=“DNA” /note=“PCR primer” /organism=“artificial sequences”
acgtggctaagaatgtcatc

SEQ ID NO: 13 - DNA, 19 bp, artificial sequences
  source 1..19: /mol_type=“DNA” /note=“PCR primer” /organism=“artificial sequences”
ctggtaggcgatgtcctta

SEQ ID NO: 14 - DNA, 18 bp, artificial sequences
  source 1..18: /mol_type=“DNA” /note=“PCR primer” /organism=“artificial sequences”
tgacgagaccaagaactg

SEQ ID NO: 15 - DNA, 18 bp, artificial sequences
  source 1..18: /mol_type=“DNA” /note=“PCR primer” /organism=“artificial sequences”
tgacgagaccaagaactg

SEQ ID NO: 16 - DNA, 20 bp, artificial sequences
  source 1..20: /mol_type=“DNA” /note=“PCR primer” /organism=“artificial sequences”
ccatccaaaccactgaaacc

SEQ ID NO: 17 - DNA, 18 bp, artificial sequences
  source 1..18: /mol_type=“DNA” /note=“PCR primer” /organism=“artificial sequences”
tcttcacaaatcctcccc

SEQ ID NO: 18 - DNA, 18 bp, artificial sequences
  source 1..18: /mol_type=“DNA” /note=“PCR primer” /organism=“artificial sequences”
tggattaaaaggacttgg

SEQ ID NO: 19 - DNA, 22 bp, artificial sequences
  source 1..22: /mol_type=“DNA” /note=“PCR primer” /organism=“artificial sequences”
gcgctggctggaaaaagagcaa

SEQ ID NO: 20 - DNA, 22 bp, artificial sequences
  source 1..22: /mol_type=“DNA” /note=“PCR primer” /organism=“artificial sequences”
tcggtgggtgctggctggtagg

SEQ ID NO: 21 - DNA, 17 bp, artificial sequences
  source 1..17: /mol_type=“DNA” /note=“PCR primer” /organism=“artificial sequences”
agccatgtacgttgcta

SEQ ID NO: 22 - DNA, 16 bp, artificial sequences
  source 1..16: /mol_type=“DNA” /note=“PCR primer” /organism=“artificial sequences”
agtccgcctagaagca

SEQ ID NO: 23 - DNA, 15 bp, artificial sequences
  source 1..15: /mol_type=“DNA” /note=“anti-mir 138” /organism=“artificial sequences”
attcacaacaccagc

SEQ ID NO: 24 - DNA, 15 bp, artificial sequences
  source 1..15: /mol_type=“DNA” /note=“antimir 138” /organism=“artificial sequences”
tgtaacacgtctata

SEQ ID NO: 25 - RNA, 23 nt, artificial sequences
  source 1..23: /mol_type=“RNA” /note=“Synthetic pre-miR-138” /organism=“artificial sequences”
agcugguguugugaaucaggccg

SEQ ID NO: 26 - DNA, 28 bp, artificial sequences
  source 1..28: /mol_type=“DNA” /note=“PCR primer” /organism=“artificial sequences”
atactcgagaaactggcccagcagtatg

SEQ ID NO: 27 - DNA, 30 bp, artificial sequences
  source 1..30: /mol_type=“DNA” /note=“PCR primer” /organism=“artificial sequences”
atagcggccgcttgcaactgaagggtgttc

(19) United States
(12) Patent Application Publication
(10) Pub. No.: US 2016/0355804 A1
(43) Pub. Date: Dec. 8, 2016

(54) MODULATION OF MICRORNA-138 FOR THE TREATMENT OF BONE LOSS

(71) Applicant: REGION SYDDANMARK, Vejle (DK)

(72) Inventors: Moustapha KASSEM, Odense M (DK); Basem ABDALLAH, Odense M (DK); Hanna TAIPALEENMAEKI, Boston, MA (US); Sakari KAUPPINEN, Smoerum (DK); Tilde ESKILDSEN, Odense C (DK); Jan STENVANG, Copenhagen K (DK)

(21) Appl. No.: 14/990,202

(22) Filed: Jan. 7, 2016

Related U.S. Application Data
(63) Continuation of application No. 14/128,792, filed on Feb. 25, 2014, now abandoned.
(60) Provisional application No. 61/500,665, filed on Jun. 24, 2011.

(51) Int. Cl. C12N 15/113

(52) U.S. Cl. CPC C12N 15/113; C12N 2310/113

(57) ABSTRACT

There is provided nucleic acids (mir-138Antimirs) for use in treating or preventing bone loss in a patient. Also there is provided a method for reducing the levels of endogenous mir-138 in a cell.

### FIELD OF THE INVENTION

The present invention relates to oligonucleotides that can be used to affect the activity of mir-138 target RNAs in order to treat bone loss disorders, such as osteoporosis.

### BACKGROUND OF THE INVENTION

A variety of disorders in humans and other mammals involve or are associated with abnormal bone resorption. Such disorders include, but are not limited to, osteoporosis, glucocorticoid-induced osteoporosis, Paget's disease, abnormally increased bone turnover, periodontitis, tooth loss, bone fractures, rheumatoid arthritis, osteoarthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption.

Bone marrow-derived human mesenchymal (stromal, skeletal) stem cells (hMSC) are a population of self-renewing, multipotent cells that have significant clinical potential in cellular therapies for tissue regeneration. hMSC can differentiate along several lineages, including the osteogenic lineage, in response to stimulation by multiple environmental factors (1-4) and it involves complex pathways regulated at both transcriptional and post-transcriptional levels. However, the regulation of these cellular pathways is not well understood. The ERK-dependent pathway plays a key role in the transcriptional control of bone formation, including phosphorylation of Runx2/Cbfa-1 (5), induction of Osterix gene expression and alkaline phosphatase activity (6, 7). Furthermore, contact with extracellular matrix (ECM) proteins can induce osteogenic differentiation of hMSC through the ERK-dependent pathway. The FAK signaling pathway is suggested to provide a link between activation of ERK1/2 by ECM proteins (7), thereby stimulating subsequent phosphorylation of Runx2/Cbfa-1, a key transcription factor controlling osteogenic gene expression (7).

MicroRNAs (miRNAs) function at the post-transcriptional level by negatively regulating translation of their target mRNAs via imperfect binding to their 3′UTRs. miRNAs have emerged as key regulators of diverse physiological and pathological processes, including cell proliferation and apoptosis (8, 9). Although 940 miRNAs have been identified in the human genome, the biological functions of relatively few miRNAs have been characterized in detail (10). The impact of miRNAs on osteoblastic differentiation of various cell types, including human adipose tissue-derived stem cells (hADSC) (11, 12), mouse mesenchymal ST2 stem cells (13), mouse premyogenic C2C12 cells (14) and mouse calvaria derived preosteoblastic cells (15, 16) has been investigated by modulation of miRNA function by antimiRs and overexpression (17). These approached successfully showed that miR-204/211 targets Runx2 in mesenchymal progenitor cells and bone marrow stromal cell (BM-MSC) and that miR-204/211 is a stimulator of adipocyte differentiation but a negative regulator of osteoblastic differentiation (18). In hADSC, miR-26a has been reported to repress the translation of the osteogenic marker, SMAD1 (11), whereas over-expression of miR-196a regulates HOXC8 and enhances osteogenic differentiation and decreases hADSC proliferation (12). miR-125b has been shown to inhibit osteoblast differentiation in the mouse mesenchymal stem cell line ST2 (13), while miR-133 and miR-135 directly target Runx2 and Smad5, respectively, and inhibit differentiation of osteoprogenitors of C2C12 mesenchymal cells (14). Moreover, miR-141 and miR-200a were recently found to be involved in pre-osteoblast differentiation through regulation of their common target DIx5 (19), whereas miR-206 inhibits osteogenesis in vitro and in vivo by targeting Cx43 (20). Additionally, miR-29b and miR-2861 were recently characterized as positive regulators by targeting inhibitors of osteoblast differentiation (15, 16).

The first generation of oligonucleotides capable of modulating the activity of a mRNA were antisense oligonucleotides. One reason for interest in such oligonucleotides is the potential for exquisite and predictable specificity that can be achieved because of specific base pairing. In other words, it is in theory very simple to design an oligonucleotide that is highly specific for a given nucleic acid, such as an mRNA.

MicroRNAs are a class of endogenous RNA molecules that has recently been discovered and function via the RNAi machinery. Currently, several hundreds human microRNAs have been discovered and the number is rapidly increasing. It is now believed that more than one third of all human genes may be regulated by microRNAs. Therefore, microRNAs themselves may be used to regulate the activity of target RNAs, and consequently e.g. be used as therapeutics.

MicroRNAs are single-stranded RNA molecules of about 19-25 nucleotides in length, which regulate gene expression. miRNAs are either expressed from non-protein-coding transcripts or mostly expressed from protein coding transcripts. They are processed from primary transcripts known as pri-miRNA to shorter stem-loop structures called pre-miRNA and finally to functional mature miRNA. Mature miRNA molecules are partially complementary to one or more messenger RNA (mRNA) molecules, and their main function is to inhibit gene expression. This may occur by preventing mRNA translation or increasing mRNA turnover/degradation.

The function of miRNAs appears to be mainly in gene regulation. For that purpose, an miRNA is (partly) complementary to a part of one or more mRNAs. Animal miRNAs are usually complementary to a site in the 3′ UTR. The annealing of the miRNA to the mRNA then inhibits protein translation, and sometimes facilitates cleavage of the mRNA (depending on the degree of complementarity). In such cases, the formation of the double-stranded RNA through the binding of the miRNA to mRNA inhibits the mRNA transcript through a process similar to RNA interference (RNAi). Further, miRNAs may regulate gene expression post-transcriptionally at the level of translational inhibition at P-bodies. These are regions within the cytoplasm consisting of many enzymes involved in mRNA turnover; P bodies are likely the site of miRNA action, as miRNA-targeted mRNAs are recruited to P bodies and degraded or sequestered from the translational machinery. In other cases it is believed that the miRNA complex blocks the protein translation machinery or otherwise prevents protein translation without causing the mRNA to be degraded. miRNAs may also target methylation of genomic sites which correspond to targeted mRNAs. miRNAs function in association with a complement of proteins collectively termed the miRNP (miRNA ribonucleoprotein complex).

A recent approach has been put forward, wherein the activity of a target RNA is regulated by inhibiting the activity of a microRNA. The microRNA can be inhibited using complementary oligonucleotides that have been termed antimirs and antagomirs. Another recent approach is the socalled blockmir, which is an oligonucleotide that is complementary to the microRNA target sequence on the mRNA it regulates. Hence, microRNA modulation may be achieved either by using antimirs or by using blockmirs.

Elucidating the mechanisms regulating human mesenchymal stem cell (hMSC) differentiation into osteogenic lineage is of importance to improve therapeutic treatments of bone loss diseases, such as osteoporosis.

### SUMMARY OF THE INVENTION

The present inventors have surprisingly found that inhibition of mir-138 by an antimir (or blockmir) oligonucleotide markedly increases osteogenic differentiation in vitro and enhanced ectopic bone formation in vivo.

Accordingly, the present invention relates to a nucleic acid capable of hybridizing to and/or inhibiting the microRNA mir-138 (AGCUGGUGUUGUGAAUCAGGCCG).

Specifically the present invention provides a method for treatment of bone loss disorders, such as osteoporosis, comprising the steps of providing one or more nucleic acids (antimir) capable of hybridizing to a mir-138, and administering said one or more nucleic acids to an individual in need thereof.

Preferably, said one or more nucleic acids comprises less than 50 nucleic acids, such as less than 45 nucleic acids, for example less than 40 nucleic acids, such as less than 35 nucleic acids, for example less than 30 nucleic acids, such as less than 25 nucleic acids, for example less than 20 nucleic acids, such as less than 15 nucleic acids, for example less than 10 nucleic acids, such as less than 8 nucleic acids, for example less than 6 nucleic acids.

In certain embodiments, the bone loss is associated with ankylosing spondylitis, renal osteodystrophy (e.g., in patients undergoing dialysis), osteoporosis, glucocorticoid-induced osteoporosis, Paget's disease, abnormally increased bone turnover, periodontitis, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions. In certain embodiments, the bone loss is associated with ankylosing spondylitis, renal osteodystrophy (e.g., in patients undergoing dialysis), osteoporosis, glucocorticoid-induced osteoporosis, Paget's disease, abnormally increased bone turnover, bone fractures, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions.

The antimir nucleic acids are preferably selected from those listed in Table 2.

The present invention also relates to the chemical modification of said nucleic acid so as to improve resistance to degradation in vivo or in vitro, improve stability, increase affinity for target, optimize solubility properties in vivo or in vitro and render the nucleotide sequence more suitable as a therapeutic agent. In one embodiment, one or more nucleobases are modified; in another embodiment, one or more backbone residues are modified; in a further embodiment, one or more internucleoside linkers of the nucleic acid according to the present invention are modified. The nucleic acids according to the present invention may be modified with one or more of said chemical modifications simultaneously. More details concerning the modifications and design rules within the ambit of the present invention can be found in inter alia the published PCT-applications WO2007090073, WO2007027894, WO2007021896, and WO2005079397, which are incorporated herein in their entirety.

The present invention also relates to the conjugation of the nucleic acid. The nucleic acid according to the present invention may be conjugated with one or more of said conjugates simultaneously.

In one embodiment, the nucleic acid capable of hybridizing to mir-138 according to the present invention inhibits said miRNA sequence.

The present invention is directed in a further aspect to a method for reducing the levels of endogenous mir-138, said method comprising the introduction of the nucleic acid and/or the vector according to the present invention into a cell in an amount sufficient to reduce the levels of said mir-138.

The present invention further discloses a method for treatment of bone loss disorders, such as osteoporosis, comprising the steps of providing one or more nucleic acids capable of hybridizing to a mir-138 or a mir-138 target site, and administering said one or more nucleic acids to an individual in need thereof. Similarly the present invention provides a nucleic acid capable of hybridizing to a mir-138 or a mir-138 target site for use in the treatment of bone disorders, such as osteoporosis.

Examples of nucleic acids (Antimirs) capable of hybridizing to a mir-138 may be found in various literature sources; Table 2 lists preferred antimirs of the present invention, which antimirs are described in more detail in the published PCT applications (see third column in Table 2), which are incorporated herein in their entirety.

TABLE 2

| SEQ ID NO | ANTIMIR SEQUENCE | REFERENCE |
|---|---|---|
| 1 | AtTcaCAacAcCaGC | WO2007112754 |
| 2 | AACACCAGC | WO2009043353 |
| 3 | GTGAAGTAG | WO2009043353 |
| 4 | GTGAAATAG | WO2009043353 |
| 5 | ACACCAGC | WO2009043353 |
| 6 | TGAAGTAG | WO2009043353 |
| 7 | TGAAATAG | WO2009043353 |
| 8 | CACCAGC | WO2009043353 |
| 9 | GAAGTAG | WO2009043353 |
| 10 | GAAATAG | WO2009043353 |
| 11 | GATTCACAACACCAGCT | WO2005013901 |

### BRIEF DESCRIPTION OF THE DRAWINGS

FIG. 1 discloses osteoblast differentiation of hMSC.

FIG. 2 shows that miR-138 inhibits osteoblast differentiation.

FIG. 3 shows that miR-138 inhibits ectopic in vivo bone formation.

FIG. 4 shows that FAK is a potential target of miR-138.

FIG. 5 shows that miR-138 targets FAK and inhibits ERK pathway.

FIG. 6 shows a proposed model for miR-138 mediated suppression of osteoblast differentiation.

FIG. 7 shows expression analysis of microRNA-138 (miR-138) and protein tyrosine kinase 2 (PTK2).

FIG. 8 shows effects of miR transfection into cells.

### DETAILED DESCRIPTION OF THE INVENTION

The term “nucleotide” refers to any of the four deoxyribonucleotides, dA, dG, dT, and dC (constituents of DNA), and the four ribonucleotides, A, G, U, and C (constituents of RNA). INA, LNA, and any other nucleic acid capable of specific base pairing are also suitable in accordance with the present invention. Each natural nucleotide comprises or essentially consists of a sugar moiety (ribose or deoxyribose), a phosphate moiety, and a natural/standard base moiety. Natural nucleotides bind to complementary nucleotides according to well-known rules of base pairing (Watson and Crick), where adenine (A) pairs with thymine (T) or uracil (U); and where guanine (G) pairs with cytosine (C), wherein corresponding base-pairs are part of complementary, anti-parallel nucleotide strands. The base pairing results in a specific hybridization between predetermined and complementary nucleotides.

Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes e.g. so-called “peptide nucleic acids,” which comprise naturally-occurring or modified nucleic acid bases attached to a polyamide backbone. Nucleic acids can be either single stranded or double stranded. In an aspect of the present invention, ‘nucleic acid’ is meant to comprise antisense oligonucleotides (ASO), small inhibitory RNAs (siRNA), short hairpin RNA (shRNA) and microRNA (miRNA).

The term Antimir (or antagomir) refers to an oligonucleotide complementary to a microRNA, such as mir-138. The term blockmir refers to an oligonucleotide that is complementary to the microRNA target sequence on the mRNA it regulates. MicroRNA modulation can be achieved either by using antimirs or by using blockmirs.

The term bone loss disorder in accordance with the present invention should be interpreted broadly. In certain embodiments, the bone loss is associated with ankylosing spondylitis, renal osteodystrophy (e.g., in patients undergoing dialysis), osteoporosis, glucocorticoid-induced osteoporosis, Paget's disease, abnormally increased bone turnover, periodontitis, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions. In certain embodiments, the bone loss is associated with ankylosing spondylitis, renal osteodystrophy (e.g., in patients undergoing dialysis), osteoporosis, glucocorticoid-induced osteoporosis, Paget's disease, abnormally increased bone turnover, bone fractures, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions. In certain embodiments, the bone loss is associated with ankylosing spondylitis, renal osteodystrophy (e.g., in patients undergoing dialysis), osteoporosis, glucocorticoid-induced osteoporosis, abnormally increased bone turnover, periodontitis, bone fractures, periprosthetic osteolysis, osteogenesis imperfecta, hypercalcemia of malignancy, multiple myeloma, bone loss associated with microgravity, Langerhan's Cell Histiocytosis (LHC), bone loss associated with renal tubular disorders, or bone loss associated with bed-ridden conditions. In certain embodiments, the bone loss is associated with osteoporosis, glucocorticoid-induced osteoporosis, Paget's disease, abnormally increased bone turnover, periodontitis, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions. In certain embodiments, the bone loss is associated with osteoporosis, glucocorticoid-induced osteoporosis, Paget's disease, abnormally increased bone turnover, bone fractures, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions. In certain embodiments, the bone loss is associated with osteoporosis, glucocorticoid-induced osteoporosis, abnormally increased bone turnover, periodontitis, bone fractures, periprosthetic osteolysis, osteogenesis imperfecta, hypercalcemia of malignancy, multiple myeloma, bone loss associated with microgravity, Langerhan's Cell Histiocytosis (LHC), bone loss associated with renal tubular disorders, or bone loss associated with bed-ridden conditions.

### EXAMPLE

With the aim to identify specific miRNAs with a potential to improve bone formation in vivo the present inventors first performed miRNA microarray to compare miRNA expression profile of undifferentiated hMSCs to hMSCs differentiated into osteogenic lineage. Microarray analysis and further validation of miRNA expression by qRT-PCR revealed significant downregulation of miR-138 during osteoblast differentiation of hMSCs.

Overexpression of miR-138 by transfection with pre-miR-138 decreased osteogenic differentiation capacity of hMSCs in vitro, demonstrated by ALP activity assay and gene expression analysis. Additionally, overexpression of miR-138 reduced ectopic bone formation in vivo by 85%. In contrast, silencing miR-138 by anti-miR-138 increased osteogenesis of hMSCs in vitro and ectopic bone formation in vivo by 60%. Target prediction analysis suggested focal adhesion kinase (FAK/Ptk2) as a potential target for miR-138. Since the FAK pathway has been reported to play a role in promoting osteoblast differentiation it is likely that miR-138 regulates bone formation by targeting Ptk2 and inhibiting FAK pathway and subsequently osteogenesis. In conclusion, the inventors have shown that miR-138 plays an important role in enhancing bone formation in vivo, possibly through inhibition of FAK signaling pathway. Inhibition of miR-138 is a new strategy for enhancing bone formation and osteoblast differentiation of MSC in vivo thereby treating osteoporosis.

Materials and Methods:

Cell Culture and Osteogenic Differentiation

Telomerase immortalized human bone marrow-derived mesenchymal stem cells (hMSC-TERT4) (3, 36) were cultured in Minimum Essential Medium (MEM) (GIBCO Invitrogen Corporation), with glutamax I (GIBCO Invitrogen Corporation) supplemented with 10% Fetal Bovine Serum (FBS) (GIBCO Invitrogen Corporation) and 1% penicillin/streptomycin (P/S) (GIBCO Invitrogen Corporation) at 37° C. in a humidified atmosphere of 5% CO₂. Cells were induced to osteoblast differentiation when cultures were 70-80% confluent. The osteoblast differentiation medium (MEM supplemented with 10% FBS, 10⁻⁸ M dexamethasone, 0.2 mM l-ascorbic acid, 10 mM β-glycerophosphate, 10 mM 1.25-vitamin-D₃, 1% P/S) was refreshed every 3 days. Cells were simultaneously cultured with MEM, 10% FBS and 1% P/S for control. The osteoblast phenotype was evaluated by determining alkaline phosphatase (ALP) activity, ALP and Alizarin Red staining, and expression of osteogenic genes by quantitative RT-PCR analysis.

Alkaline Phosphatase Staining

For detection of ALP, cells were washed with PBS, fixed with acetone/citrate buffer (Centralapoteket) pH 4.2 for 5 minutes at room temperature and stained with Naphtol-AS-TR-phosphate solution for 1 hour at room temperature. Naphtol-AS-TR-phosphate solution consist of Naphtol-AS-TR-phosphate (Sigma) diluted 1:5 in H₂O and Fast Red TR (Sigma) dissolved in 0.1 M Tris buffer (Bie and Berntsen), pH 9.0, in the ratio 1.2:1. The solutions were mixed 1:1. Cells were rinsed with destilled H₂O (dH₂O) and counterstained with Mayers-Hematoxylin (Bie and Berntsen) for 5 minutes at room temperature.

Alizarin Red Staining

Alizarin Red staining was performed to detect matrix mineralization. Cells were fixed with 70% ice-cold Ethanol for 1 h at −20° C. and stained with 40 mM alizarin red S (AR-S; Sigma-Aldrich Corp.), pH 4.2 for 10 min at room temperature.

Alkaline Phosphatase Quantification

Alkaline phosphatase activity was quantified as previously described (37). Briefly, cells were cultured on 96-well plates at a density of 10 000 cells/cm² and induced to osteogenic differentiation as described above. Cells were rinsed with TBS and fixed in 3.7% formaldehyde-90% ethanol for 30s at room temperature. Cells were incubated for 20 min at 37° C. with reaction substrate solution (1 mg/ml P-nitrophenylphosphate in 50 mM NaHCO₃, pH 9.6, 1 mM MgCl₂), and absorbance was measured at 405 nm using Elisa-reader.

Microarray and Data Processing

Total RNA was extracted using TRIzol-phenol-choroform method (Invitrogen) according to the manufacturer's protocol. RNA concentration was measured on a ND-1000 spectrophotometer (NanoDrop® Technologies) and quality of total RNA was determined on an Agilent 2100 bioanalyzer (Agilent Technologies).

MicroRNA microarray was performed at Molecular Medicine Partnership Unit (MMPU), University of Heidelberg, Germany, based on Exiqon's microarray platform (version 8.0) with LNA capture probes in quadruplicates. Microarray probes were LNA-modified oligonucleotide (miRCURY Exiqon) capture probes with sequence complementary to miRNAs. Slides were scanned using Genepix 4000B laser scanner (Axon instruments). Artifact-associated spots were eliminated by software (TIGR spotfinder 3.1.1). Image intensities were measured as a function of the median of foreground minus background. Negative values and values below 50 were normalized to one. Further data analysis was performed using Microsoft Excel with Significant Analysis of Microarrays (SAM) excel software using multiclass response dataset analysis. The data was normalized using the Limma package for statistical programming language R (version 2.5.1). Medians of four background-corrected replicas for each miRNA capture probe were uploaded into the microarray analysis software for more advanced analysis

Quantitative RT-PCR (qRT-PCR) Analysis

For qRT-PCR analysis of mRNA expression total RNA was extracted using Trizol reagent (Invitrogen) according to manufacturer's protocol. cDNA was prepared using revertAid H minus first strand cDNA synthesis kit (Fermentas). SYBR green qRT-PCR was performed using the primers ALP forward (5′-ACGTGGCTAAGAATGTCATC-3′), ALP reverse (5′-CTGGTAGGCGATGTCCTTA-3′), COL1A1 forward (5′-TGACGAGACCAAGAACTG-3′), COL1A1 reverse (5′-CCATCCAAACCACTGAAACC-3′), RUNX2 forward (5′-TCTTCACAAATCCTCCCC-3‘), RUNX2 reverse (5′-TGGATTAAAAGGACTTGG-3′), PTK2 forward (5′-GCGCTGGCTGGAAAAAGAGCAA-3′), PTK2 reverse (5′-TCGGTGGGTGCTGGCTGGTAGG-3′), β-ACTIN forward (5′-AGCCATGTACGTTGCTA-3′) and β-ACTIN reverse (5′-AGTCCGCCTAGAAGCA-3′). Expression levels were analyzed by qRT-PCR (SYBR Green supermix and iCycler IQ detection system, Bio-Rad) using conventional protocols. qRT-PCR consisted of 40 cycles (95° C. for 30 seconds, 60° C. for 30 seconds and 72° C. for 1 min) after an initial denaturation step (95° C. for 3 min). qRT-PCR products were quantified by comparative Ct (ΔΔCt) method.

For miRNA qRT-PCR, total RNA was extracted using Trizol reagent (Invitrogen). Primers specific for human miR-26a, miR-26b, miR-30c, miR-101, miR-138, miR-143, miR-222 and internal control snoRNU44 were purchased from Applied Biosystems. Amplification and detection were performed using 7500HT Fast Real-Time PCR System (Applied Biosystems), using 40 cycles of denaturation at 95° C. (10 s) and annealing/extension at 60° C. (60 s). This was preceded by reverse transcription at 42° C. for 30 min and denaturation at 85° C. for 5 min.

Transfection of Oligonucleotides

LNA oligonucleotides were synthesized as unconjugated LNA/DNA mixmers with a complete phosphorothioate backbone (IDT, USA). The antimiR-control (miR-C) was purchased from IDT, USA. LNA modified antimiRs sequences: AntimiR-138 3′-AtTcaCAacAcCaGC-5′ and AntimiR-C 3′-TgtAacAcGTcTAtA-5′, where uppercase letters refers to LNA and lowercase letters refers to DNA. Synthetic pre-miR-138 sequence 3′-AGCUGGUGUUGUGAAUCAGGCCG-5′ was RNA oligonucleotides. Transfections of 25 nM antimiR oligonucleotide (IDT, USA) or pre-miR (Ambion) with lipofectamine 2000 (Invitrogen) were performed according to the manufacturer's instructions. Cells were seeded in 6-well plates and transfections were performed when cells reached 60-70% confluence. One hour prior transfection, the medium was changed to 1 ml OPTI-MEM I Reduced Serum (Invitrogen). Cells were incubated with transfection medium, which was changed four hours after transfection to normal MEM medium with 10% FBS and 1% P/S. The transfection efficiency was evaluated by transfection of a 5′ FAM-labeled LNA oligonucleotide.

Western Blot Analysis

hMSCs were lysed by RIPA buffer (Sigma-Aldrich Corp.) and total protein concentration was determined with Pierce Coomassie Plus Bradford assay kit (Thermo Fisher Scientific Inc.). Proteins were separated by 10% SDS-PAGE and electrotransferred into nitrocellulose filters. After blocking with 5% not-fat milk for 1h at room temperature, membranes were incubated with primary antibodies against anti-rabbit FAK and pFAK (Santa Cruz Biotechnology) and anti-mouse ERK pERK and anti-rabbit α-tubulin (Cell signaling) overnight at 4° C. Membranes were incubated with horseradish-peroxidase (HRP) conjucated anti-mouse or anti-rabbit secondary antibody for 45 min at RT and protein bands were visualized with Amersham ECL chemiluminescence detection system (GE Healthcare Bio-Sciences Corp.).

Dual Luciferase Reporter Gene Construct

A 655 bp fragment of the FAK (PTK2) 3′UTR, containing the predicted binding site for hsa-miR-138, was amplified from human genomic DNA using primers with a short extension, containing cleavage sites for XhoI (5′-end) and NotI (3′-end). PTK2 forward (5′-ATACTCGAGAAACTGGCCCAGCAGTATG-3′), PTK2 reverse (5′-ATAGCGGCCGCTTGCAACTGAAGGGTGTTC-3′). Amplicons were cleaved with XhoI and NotI and cloned in between the XhoI and NotI cleavage sites of the PsiCHECK™-2 vector (Promega) downstream of the Renilla luciferase reporter gene.

Luciferase Assay

Huh7 cells were grown to 85-90% confluence in white 96 well plates in Dulbecco's Modified Eagle Medium (DMEM) (Invitrogen) supplemented with 10% FBS, 1% non-essential amino acids, L-glutamine and Penicillin/Straptamicin, at 37° C. under 5% CO₂. Cells were transfected with 20 ng of either the empty PsiCHECK™-2 vector, or the PsiCHECK™-2-PTK2 3′ UTR reporter, for four hours in reduced-serum and antibiotics-free Opti-MEM (Invitrogen) with Lipofectamine 2000 (Invitrogen). Cells were co-transfected with the pre-miR-138 or a negative control (miR-C) (Applied Biosystems) at concentrations of 0, 10 or 20 nM. After transfection, Opti-MEM was replaced by normal culture medium and cells were incubated for 24 hours. Firefly and Renilla luciferase were measured in cell lysates according to manufacturer's protocol using a Dual-Luciferase Reporter Assay System (Promega) on a Fusion™ plate reader (Perkin Elmer). Firefly luciferase activity was used for normalization and as an internal control for transfection efficiency.

Ectopic In Vivo Bone Formation Assay of Transfected hMSC Cells

hMSCs were transfected as described above, loaded on hydroxyapatite/tricalcium phosphate (HA/TCP) ceramic powder (Zimmer Scandinavia) and implanted subcutaneously into 8-week old NOD/MrkBom Tac-Prkdc^{scid} mice (Taconic) as previously described (22, 23). Briefly, cells (5×10⁵) were resuspended in 500 μL medium, transferred to 40 mg of wetted HA/TCP and incubated at 37° C. overnight. The following day, cells loaded on HA/TCP vehicles were implanted subcutaneously on the dorsal surface of the NOD/SCID mice. Each mouse received four identical implants, two on each side. Mice were anaesthetized by intra-peritoneal injection of Ketaminal® (Ketamine 100 mg/kg) (Intervet) and Rampun® (xylazine, 10 mg/kg) (Bayer HealthCare). After the surgery, mice received a subcutaneous injection of Temgesic® (buprenorphin, 0.3 mg/mL) (Schering-Plough) for pain relief.

Implants were removed after 1 or 8 weeks. One-week implants were subjected to RNA extraction while 8-week implants were fixed in 4% paraformaldehyde (Bie & Berntsen), decalcified in formic acid (Local Pharmacy, Odense University Hospital) and embedded in paraffin using conventional histopathologic methods. Samples were cut into 4 μm sections and stained with Hematoxylin and Eosin Y (Bie & Berntsens Reagent laboratories). Total bone volume per total volume was quantified as previously described (23). Four implants per treatment were engrafted into mice and three sections of each implant were quantified to minimize variation within the implants.

miRNA Target Site Prediction

A search for predicted target mRNAs was performed using the databases TargetScan and PicTar. TargetScan requires perfect complementarity with a miRNA seed sequence, whereas PicTar allows for targets with imperfect seed matches given that they pass a defined binding-energy threshold. Additionally, PicTar implements a maximum likelihood approach to incorporate the combinatorial nature of miRNA targeting (38).

Sequence conservation was examined using UCSC genome browser (http://genome.ucsc.edu/)

Statistical Analysis

Data are presented as mean±SD. Comparisons were made by using a two-tailed t-test or 1-way ANOVA for experiments with more than two subgroups. Probability values were considered statistically significant at p<0.05.

Results

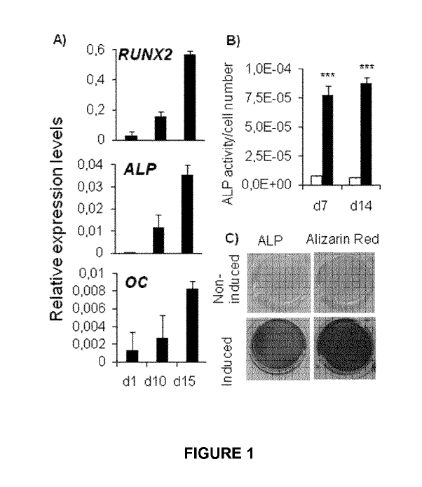

In FIG. 1 telomerized MSCs (hMSC-Tert4) were induced to osteoblast differentiation. A) Osteoblast differentiation confirmed by qRT-PCR analysis of osteoblast marker genes (Runx2, ALP and OC normalized to β-actin). B) ALP activity was measured during the course of differentiation. White bars represent non-induced and black bars represent induced samples. *** p<0.001 between non-induced and induced sample. C) ALP and Alizarin Red staining were performed at day 15. n=3 for all experiments.

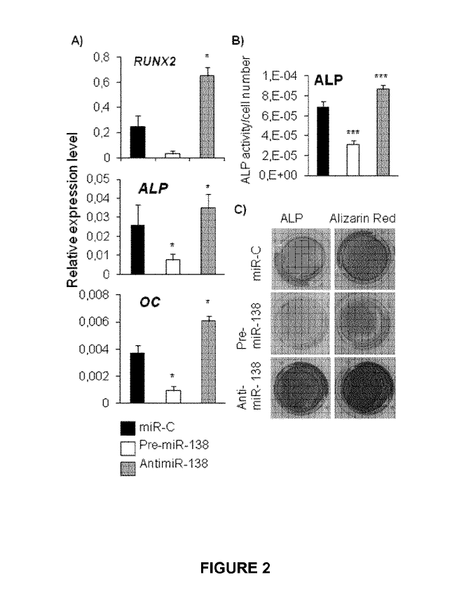

In FIG. 2 the effect of miR-138 on osteoblast differentiation was studied. hMSCs transfected with 25 nM miR-C, pre-miR-138 or antimiR-138 were induced into osteoblast differentiation for 15 days. A) Osteoblast differentiation was evaluated with qRT-PCR analysis of osteoblast marker genes (RUNX2, ALP and OC, normalized to β-actin) at day 15. B) Alkaline phosphatase activity was measured at day 10 of osteoblast differentiation. C) Alkaline phosphatase and Alizarin red staining were performed at day 15. *p<0.05, **p<0.01, ***p<0.001, n=3 for all experiments.

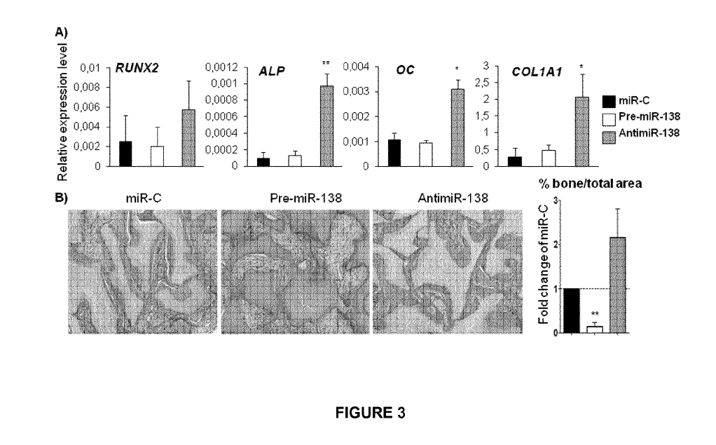

In the experiment of FIG. 3 hMSC were transfected with 25 nM miR-C, pre-miR-138 or antimir-138 and implanted into NOD/SCID mice. A) qRT-PCR analysis of osteoblast genes (RUNX2, COL1A1, ALP and OC normalized to (3-actin) was performed after 1 week of implantation. B) H&E staining was performed after 8 weeks of implantation. Bone formation was quantified as total bone volume per total volume from H&E staining and expressed as fold change of miR-C. *p<0.05, **p<0.01, four implants per treatment were engrafted into mice and three sections of each implant were quantified to minimize variations within the implants.

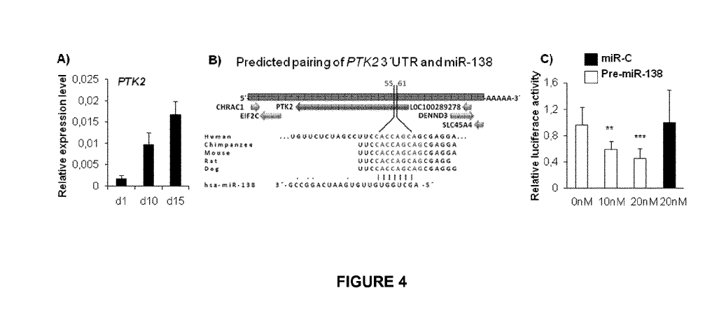

In the experiment of FIG. 4 A) PTK2 gene expression profile during osteoblast differentiation of hMSC was quantified with qRT-PCR. B) Computational analysis was performed for the complementarily of miR-138 seed sequence to the 3′UTR of PTK2 and conservation of the putative binding site in vertebrates. C) Huh7 cells were transfected with 20 ng of either the empty PsiCHECK™-2 vector, or the PsiCHECK™-2-PTK2 vector. Cells were co-transfected with 0, 10 or 20 nM of the pre-miR-138 or a negative control. Firefly and Renilla luciferase were measured in cell lysates and values are normalized to the PsiCHECK vector and presented as fold change of miR-C. **p<0.01, ***p<0.001

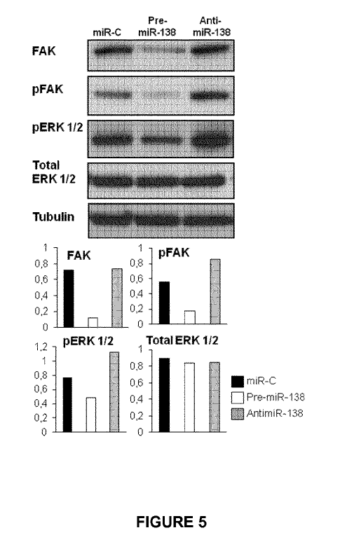

In the experiment of FIG. 5 hMSC were transfected with 25 nM miR-C, pre-miR-138 or antimir-138 and induced to osteoblast differentiation and western blot analysis for FAK protein, phosphorylation of FAK and ERK1/2 were performed at day 2. Graphs represent quantifications of western blot results.

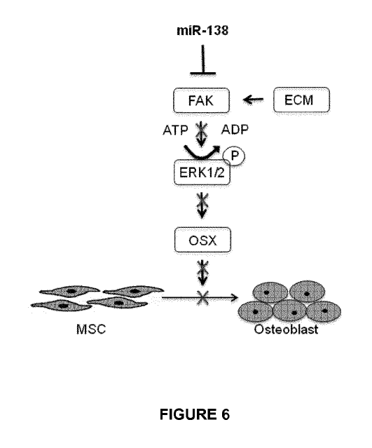

In FIG. 6 miR-138 is expressed in undifferentiated MSC and suppresses FAK translation, thereby decrease phosphorylation of FAK and its downstream target ERK1/2. Subsequently, inhibition of the cascade results in decreased expression of OSX and suppression of osteoblast differentiation of MSC.

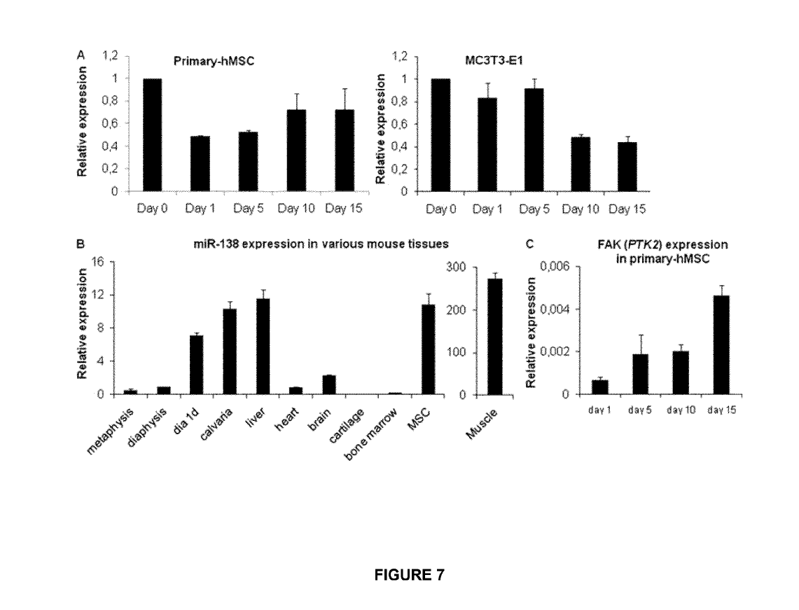

In FIG. 7 there is shown an expression analysis of microRNA-138 (miR-138) and protein tyrosine kinase 2 (PTK2). (A) Expression of miR-138 was measured in primary human stromal (mesenchymal) stem cells (hMSCs) and calvarial preosteoblastic MC3T3-E1 cells during osteoblast differentiation. (B) miR-138 expression was evaluated in 5-d-old mouse tissues. (C) Expression of focal adhesion kinase (FAK; PTK2) in primary hMSCs. Expression of PTK2 in primary hMSCs during osteoblast differentiation evaluated at day 1, 5, 10, and 15 by quantitative RT-PCR (qRT-PCR).

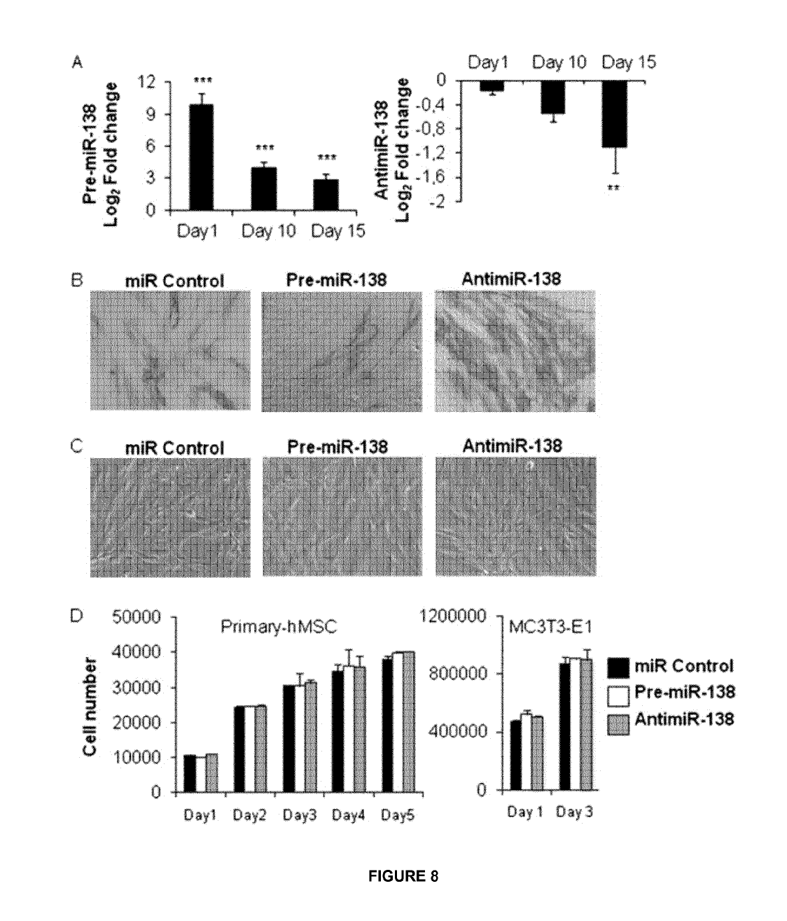

In FIG. 8 there is shown effects of miR transfection into cells. (A) Expression of miR-138 in transfected hMSCs. hMSCs were transfected with miR-C, premiR-138, and antimiR-138 and induced to osteoblast differentiation. The miR-138 expression levels in premiR-138—(Left) and antimiR-138—transfected (Right) hMSCs were quantified by qRT-PCR and normalized to sn/snoRNU44, and values are presented as log2 fold change over miR-C. **P<0.01; ***P<0.001 (n=3). (B) Osteoblast differentiation of miR-138—transfected primary hMSCs. Primary hMSCs were transfected with miR-C, premiR-138, and antimiR-138 and induced to osteoblast differentiation. Differentiation was evaluated at day 15 by alkaline phosphatase (ALP) staining. (C) Morphology of miR-138—tranfected hMSCs. Morphology was evaluated by photography of primary hMSCs transfected with miR-C, premiR-138, and antimiR-138 under the microscope at day 1. (D) Proliferation of miR-138—tranfected primary hMSCs and MC3T3-E1 cells. Proliferation was evaluated by cell count in primary hMSCs and MC3T3 calvarial cells transfected with miR-C, premiR-138, and antimiR-138.

Identification of Differentially Expressed miRNAs During Osteoblast Differentiation

Osteoblast differentiation of hMSC was induced by using standard osteoblast-induction medium containing dexamethasone, ascorbic acid, β-glycerolphosphate and vitamin-D₃ and evidenced by increased expression of genes associated with osteoblast differentiation; RUNX2, alkaline phosphatase (ALP) and osteocalcin (OC) at day 1, 10 and 15 after induction (FIG. 1A). The osteoblast phenotype was also confirmed by demonstration of increased alkaline phosphatase activity (FIGS. 1B and C) and Alizarin Red staining for matrix mineralization (FIG. 1C). Increased expression of osteoblast-associated genes and the observed osteoblast phenotype were in accordance with previous reports describing hMSC differentiation into osteoblasts (1-3).

To identify differentially expressed miRNAs during osteoblast differentiation we carried out miRNA array profiling of hMSC 10 days after induction to osteoblasts using LNA microarrays (24). Expression levels of 33 miRNAs were significantly altered between differentiated and non-differentiated cells. Of these, 7 miRNAs were selected for validation by qRT-PCR, based on their relative difference score and up- or down-regulation (SI Table 1). Microarray analysis and qRT-PCR data showed that the expression of miR-26a, 26b, 30c, 101 and 143 were up-regulated and miR-138 and 222 were down-regulated during osteoblast differentiation of hMSCs.

Effect of miR-138 on the Osteoblast Differentiation of hMSC

To evaluate the biological effect of the differentially expressed miRNAs on osteogenesis, antimiRs and miRNA over-expression for selected miRNAs were applied in a pilot functional screening for the impact on osteoblast differentiation in vitro and ectopic bone formation in vivo (data not shown) and pointed out to miR-138 as a novel negative regulator of osteoblastic differentiation. We over-expressed or inhibited miR-138 levels utilizing synthetic pre-miR and LNA-modified anti-oligonucleotides designed as complementary sequences to mature miRNAs, so-called antimiRs. AntimiR oligonucleotides can be used as specific inhibitors of miRNA function, thus providing a valuable tool to access the biological function of specific miRNAs in vitro and in vivo (25, 26). Transfection efficiency of 80% was observed using a 5′ FAM-labeled control LNA oligonucleotide (data not shown). The degree of miRNA inhibition and over-expression was monitored by qRT-PCR after transfection of antimiR-138 or pre-miR-138 to hMSCs at 25 nM, respectively. The mature miR-138 levels were elevated ˜900-fold relative to control-treated cells 24 hours post transfection, with the levels still being 16- and 8-fold higher than in the control 10 and 15 days after pre-miR-138 transfection, respectively (FIG. 7). By comparison, treatment of hMSCs with antimiR-138 led to inhibition of miR-138 by 1.1-, 1.5- and 2-fold 1, 10 and 15 days after transfection (FIG. 7).

To study the impact of miR-138 on hMSC osteoblast differentiation, hMSCs were induced to differentiate to osteoblasts after transfection with either pre-miR-138 or antimiR-138. Inhibition of miR-138 significantly enhanced osteogenic differentiation, as indicated by higher expression of the osteoblast-specific genes RUNX2, ALP and OC and (FIG. 2A), and increased ALP activity and enhanced in vitro matrix mineralization visualized by Alizarin red staining (FIGS. 2B,C) in antimiR-138 transfected hMSCs as compared to control transfected cells. In contrast, ALP activity, matrix mineralization and the osteoblast marker gene expression were reduced in pre-miR-138 transfected hMSCs (FIGS. 2A,B,C). Taken together, our results indicate that miR-138 is a negative regulator of osteoblast differentiation of hMSCs.

Effect of miR-138 on Ectopic In Vivo Bone Formation

To study whether silencing of miR-138 enhances ectopic bone formation also in vivo, untransfected control hMSCs and hMSCs transfected with miR-138, antimir-138 or miR-control were loaded on hydroxyapatite implants in NOD/SCID mice for 1 or 8 weeks. No major changes were observed by histology of the implants with untransfected hMSCs compared to miR-C transfected hMSCs. Gene expression of osteoblast marker genes was analyzed after one week of implantation. qRT-PCR analysis revealed up-regulation of RUNX2, ALP, COL1A1 and OC in the antimiR-138 treated implants as compared to implants transfected with miR-C (FIG. 3A), corroborating the results obtained from in vitro cell culture assays. Additionally, we determined the ability of miR-138 inhibition or over-expression to regulate ectopic bone formation in vivo by quantifying the area of bone per total area after 8 weeks. Bone formation was increased 2.2-fold in implants treated with the antimiR-138 compared to miR-C (FIG. 3B), indicating that inhibition of miR-138 enhances bone formation of hMSC. Furthermore, over-expression of miR-138 decreased bone formation by 6.7 fold, compared to miR-C implants (FIG. 3B), supporting the notion that miR-138 negatively regulates osteoblast differentiation and bone formation in vivo.

miR-138 Targets FAK and Regulates FAK Downstream Signaling

To understand the molecular mechanisms that underlie miR-138 -mediated regulation, we searched for potential targets of miR-138 implicated in osteoblast differentiation using the miRNA target prediction algorithms TargetScan and PicTar (27). Among the predicted targets we identified focal adhension kinase (FAK), which provides a link between activation of ERK1/2 and stimulation of the Runx2/Cbfa1 transcription factor. To confirm the involvement of FAK in osteogenesis of hMSC we studied the expression pattern of FAK during differentiation. qRT-PCR analysis revealed that expression of PTK2, the gene encoding FAK, was increased during osteoblast differentiation similar to osteoblast marker genes and coinciding with down-regulation of miR-138 (FIG. 4A). According to in silico analysis, PTK2 has a 7 nucleotide seed match site for miR-138 within its 3′UTR, and this putative target site is highly conserved among the vertebrates (FIG. 4B).

To determine whether miR-138 inhibits PTK2 gene expression by binding to the predicted target site in the 3′UTR, we used a dual luciferase reporter gene system (28), with Renilla luciferase as a reporter gene and Firefly luciferase as an internal control. Assays were performed in Huh7 cells that were chosen based on their low endogenous expression of miRNAs (29). Co-transfection of the PTK2 3′UTR luciferase reporter with pre-miR-138 resulted in concentration-dependent down-regulation of luciferase activity, compared to the mock or scrambled oligonucleotide controls (miR-C) (FIG. 4C). In comparison, pre-miR-138 had no effect on the luciferase control reporter without the PTK2 3′UTR, implying that PTK2 is a direct target of miR-138.

Gene expression analysis revealed no significant change in PTK2 mRNA levels when miR-138 was either over-expressed or antagonized (FIG. 8a). However, Western blot analysis showed reduced FAK protein levels in the pre-miR-138 treated cells at day 2 compared to miR-C transfected samples (FIG. 5). Since the FAK signaling pathway is suggested to provide a link between activation of ERK1/2 by ECM proteins in osteogenesis (7), we next assessed phosphorylation of FAK and ERK1/2. Western blot analysis showed markedly decreased phosphorylation of both FAK and ERK1/2 in miR-138 over-expressing hMSCs, while levels of pFAK and pERK1/2 were increased in the antimiR-138 transfected hMSC (FIG. 5). Accordingly, expression of osterix (osx), a downstream target gene of the ERK1/2 pathway (7), was decreased when miR-138 was over-expressed and increased in the absence of miR-138 (FIG. 8b), which is consistent with the notion that miR-138 suppresses FAK downstream signaling by negatively regulating FAK at the post-transcriptional level.

Discussion

Bone marrow contains a population of stromal (skeletal, mesenchymal) stem cells (hMSC) that under appropriate in vivo and in vitro conditions can differentiate into osteoblastic cells (30). Differentiation of hMSC into osteoblastic cells is a highly regulated process involving complex pathways and de-regulation may lead to pathological conditions. Therefore the molecular mechanisms of this process needs to be explored to ultimately improve therapies for the related diseases. Recently, it has been shown that miRNAs influence the complexity of the “sternness state” in a number of cellular compartments through negative regulation of gene expression at the post-transcriptional level (31).

The present inventors have identified miR-138 as a negative regulator of hMSC osteoblast differentiation and demonstrated that antimiR mediated silencing of miR-138 significantly enhance ectopic bone formation in vivo. This suggests functional silencing of miR-138 as a potential novel strategy for anabolic treatment of osteoporosis. The therapeutic feasibility of such an approach has been demonstrated in the work by Li and co-workers who demonstrated that a single tail vein injection of antimiR-2861 caused minimum femur bone mineral density in ovarectomized mice (15).

The present inventors conducted genome-wide array analysis of the miRNA levels during osteogenic differentiation of hMSC and found deregulation of several miRNAs during osteoblast differentiation of hMSC in vitro. More detailed analysis suggested miR-138 as a novel negative regulator of osteoblastic differentiation. In vitro experiment revealed that inhibition of miR-138 function enhanced osteoblast differentiation, whereas miR-138 over-expression inhibited the osteogenic potential. These experiments were extended to an in vivo milieu where silencing of miR-138 by antimiR-138 led to increased ectopic bone formation while over-expression of miR-138 significantly diminished bone formation. These findings suggest that miR-138 plays a pivotal role in bone formation in vivo by negative regulation of osteogenic differentiation in hMSCs leading to reduced ectopic bone formation.

Recently, miR-138 has been implicated in differentiation of human adipose tissue-derived mesenchymal stem cells (hAD-MSCs) and miR-138 was found to be a negative regulator of adipocyte differentiation (21). These new data, together with our findings suggests a general role of miR-138 as an inhibitor of hMSC differentiation and maintenance of the “sternness state”.

To study the molecular mechanism whereby miR-138 regulates osteogenesis, the present inventors searched for potential target genes that have an established function in promoting osteogenesis. Interestingly, the 3′UTR of PTK2 possess a seven nucleotides perfect match site to the miR-138 seed region. The protein encoded by PTK2, FAK, has been shown to function as an activator of extracellular signal-related kinase (ERK1 and ERK2) via the Grb2-Sos-Ras pathway during osteogenic differentiation of hMSC (32, 33). Recently, miR-138 was found to target RhoC and ROCK (34), which are also involved in regulation of osteoblast differentiation and this may be an additional mechanism for miR-138 as negative modulator of osteoblast differentiation. However, the exact organization of these pathways in developing bone is not well understood, although osteoblasts in osteoporosis and osteoarthritis patients have reduced FAK activity (35).

Here, the present inventors show that miR-138 over-expression results in down-regulation of FAK at the protein level, whereas functional inhibition of miR-138 by antimiR-138 leads to de-repression of FAK, strongly suggesting that FAK is regulated by miR-138 during osteogenesis. Indeed, PTK2 3′UTR luciferase reporter assays confirmed that FAK is a direct target of miR-138. Furthermore, over-expression of miR-138 decreased phosphorylatation of FAK and subsequently attenuated activation of FAK downstream signaling, as shown by decreased phosphorylation of ERK1/2 in hMSCs. Activation of ERK1/2 pathway has emerged as an important regulator of osteoblast differentiation, where it regulates Runx2 phosphorylation and subsequently expression of Osterix (7).

Significant up-regulation of Osterix in the absence of miR-138 supports the hypothesis that inhibition of osteoblast differentiation by miR-138 is due to suppression of the downstream pathway of FAK (FIG. 6).

The impact of miRNAs on osteoblastic differentiation of a number of cell types has been investigated by modulation of miRNA function by antimiRs and over-expression. These approaches have successfully demonstrated that miR-204/211 targets Runx2, stimulates adipocyte differentiation and diminishes osteoblastic differentiation (18). Employing similar approach, enhanced activity of miR-125b in mouse ST2 cells inhibits osteoblastic differentiation (13) whereas miR-2861 acts as a positive regulator by targeting HDAC5 (15). Luzi et al. (11) showed that miR-26a expression was increased hADSC differentiation, where expression of SMAD1 was inversely correlated to that of miR-26a, suggesting the presence of a negative regulatory mechanism in late osteogenic differentiation of hASC. Moreover Kim et al. (12) demonstrated an increase in miR-196a expression and concomitant decrease of HOX8 expression, a negative regulator of SMAD1, during osteogenic differentiation of hASCs.

The present inventors found that miR-138 repress FAK expression, which, in turn, results in suppression of the FAK-ERK1/2 signaling pathway. Importantly, our results show that functional inhibition of miR-138 can accelerate osteogenic differentiation of hMSCs leading to increased bone formation in vivo, suggesting that therapeutic approaches targeting miR-138 could be useful in the enhancing bone formation and treatment of pathological conditions of bone loss.

### REFERENCES

1. Jaiswal, R. K., Jaiswal, N., Bruder, S. P., Mbalaviele, G., Marshak, D. R., & Pittenger, M. F. (2000) J. Biol. Chem. 275, 9645-9652.
2. Pittenger, M. F., Mackay, A. M., Beck, S. C., Jaiswal, R. K., Douglas, R., Mosca, J. D., Moorman, M. A., Simonetti, D. W., Craig, S., & Marshak, D. R. (1999) Science 284, 143-147.
3. Abdallah, B. M., Haack-Sorensen, M., Burns, J. S., Elsnab, B., Jakob, F., Hokland, P., & Kassem, M. (2005) Biochem. Biophys. Res. Commun. 326, 527-538.
4. Abdallah, B. M. & Kassem, M. (2008) Gene Ther. 15, 109-116.
5. Xiao, G., Jiang, D., Thomas, P., Benson, M. D., Guan, K., Karsenty, G., & Franceschi, R. T. (2000) J. Biol. Chem. 275, 4453-4459.
6. Takeuchi, Y., Suzawa, M., Kikuchi, T., Nishida, E., Fujita, T., & Matsumoto, T. (1997) J. Biol. Chem. 272, 29309-29316.
7. Salasznyk, R. M., Klees, R. F., Williams, W. A., Boskey, A., & Plopper, G. E. (2007) Exp. Cell Res. 313, 22-37.
8. Brennecke, J., Hipfner, D. R., Stark, A., Russell, R. B., & Cohen, S. M. (2003) Cell 113, 25-36.
9. Xu, P., Vernooy, S. Y., Guo, M., & Hay, B. A. (2003) Curr. Biol. 13, 790-795.
10. Ambros, V. (2004) Nature 431, 350-355.
11. Luzi, E., Marini, F., Sala, S. C., Tognarini, I., Galli, G., & Brandi, M. L. (2008) J. Bone Miner. Res. 23, 287-295.
12. Kim, Y. J., Bae, S. W., Yu, S. S., Bae, Y. C., & Jung, J. S. (2009) J. Bone Miner. Res. 24, 816-825.
13. Mizuno, Y., Yagi, K., Tokuzawa, Y., Kanesaki-Yatsuka, Y., Suda, T., Katagiri, T., Fukuda, T., Maruyama, M., Okuda, A., Amemiya, T. et al. (2008) Biochem. Biophys. Res. Commun. 368, 267-272.
14. Li, Z., Hassan, M. Q., Volinia, S., van Wijnen, A. J., Stein, J. L., Croce, C. M., Lian, J. B., & Stein, G. S. (2008) Proc. Natl. Acad. Sci. U.S.A 105, 13906-13911.
15. Li, H., Xie, H., Liu, W., Hu, R., Huang, B., Tan, Y. F., Xu, K., Sheng, Z. F., Zhou, H. D., Wu, X. P. et al. (2009) J. Clin. Invest 119, 3666-3677.
16. Li, Z., Hassan, M. Q., Jafferji, M., Aqeilan, R. I., Garzon, R., Croce, C. M., van Wijnen, A. J., Stein, J. L., Stein, G. S., & Lian, J. B. (2009) J. Biol. Chem. 284, 15676-15684.
17. Hu, R., Li, H., Liu, W., Yang, L., Tan, Y. F., & Luo, X. H. (2010) Expert. Opin. Ther. Targets. 14, 1109-1120.
18. Huang, J., Zhao, L., Xing, L., & Chen, D. (2010) Stem Cells 28, 357-364.
19. Itoh, T., Nozawa, Y., & Akao, Y. (2009) J. Biol. Chem. 284, 19272-19279.
20. Inose, H., Ochi, H., Kimura, A., Fujita, K., Xu, R., Sato, S., Iwasaki, M., Sunamura, S., Takeuchi, Y., Fukumoto, S. et al. (2009) Proc. Natl. Acad. Sci. U.S.A.
21. Yang, Z., Bian, C., Zhou, H., Huang, S., Wang, S., Liao, L., & Zhao, R. C. (2010) Stem Cells Dev.
22. Stenderup, K., Rosada, C., Justesen, J., Al-Soubky, T., gnaes-Hansen, F., & Kassem, M. (2004) Biogerontology. 5, 107-118.
23. Abdallah, B. M., Ditzel, N., & Kassem, M. (2008) Methods Mol. Biol. 455, 89-100.
24. Castoldi, M., Schmidt, S., Benes, V., Noerholm, M., Kulozik, A. E., Hentze, M. W., & Muckenthaler, M. U. (2006) RNA. 12, 913-920.
25. Elmen, J., Lindow, M., Schutz, S., Lawrence, M., Petri, A., Obad, S., Lindholm, M., Hedtjarn, M., Hansen, H. F., Berger, U. et al. (2008) Nature 452, 896-899.
26. Petri, A., Lindow, M., & Kauppinen, S. (2009) Cancer Res. 69, 393-395.
27. Lewis, B. P., Shih, I. H., Jones-Rhoades, M. W., Bartel, D. P., & Burge, C. B. (2003) Cell 115, 787-798.
28. Kuhn, D. E., Martin, M. M., Feldman, D. S., Terry, A. V., Jr., Nuovo, G. J., & Elton, T. S. (2008) Methods 44, 47-54.
29. Landgraf, P., Rusu, M., Sheridan, R., Sewer, A., lovino, N., Aravin, A., Pfeffer, S., Rice, A., Kamphorst, A. O., Landthaler, M. et al. (2007) Cell 129, 1401-1414.
30. Friedenstein, A. J., Chailakhjan, R. K., & Lalykina, K. S. (1970) Cell Tissue Kinet. 3, 393-403.
31. Oakley, E. J. & Van, Z. G. (2007) Leukemia 21, 612-621.
32. Schaller, M. D. (2001) Biochim. Biophys. Acta 1540, 1-21.
33. Schlaepfer, D. D., Hanks, S. K., Hunter, T., & van der, G. P. (1994) Nature 372, 786-791.
34. Jiang, L., Liu, X., Kolokythas, A., Yu, J., Wang, A., Heidbreder, C. E., Shi, F., & Zhou, X. (2010) Int. J. Cancer 127, 505-512.
35. Perinpanayagam, H., Zaharias, R., Stanford, C., Brand, R., Keller, J., & Schneider, G. (2001) J. Orthop. Res. 19, 993-1000.
36. Simonsen, J. L., Rosada, C., Serakinci, N., Justesen, J., Stenderup, K., Rattan, S. I., Jensen, T. G., & Kassem, M. (2002) Nat. Biotechnol. 20, 592-596.
37. Qiu, W., Hu, Y., Andersen, T. E., Jafari, A., Li, N., Chen, W., & Kassem, M. (2010) J. Biol. Chem. 285, 14438-14449.
38. Sethupathy, P., Megraw, M., & Hatzigeorgiou, A. G. (2006) Nat. Methods 3, 881-886.

1. Nucleic acid (Antimir) for use in treating or preventing bone loss in a patient, said antimir capable of hybridizing to a mir-138 (AGCUGGUGUUGUGAAUCAGGCCG).

2. Nucleic acid (Antimir) for use according to claim 1, wherein said nucleic acid comprises less than 50 nucleic acids, such as less than 45 nucleic acids, for example less than 40 nucleic acids, such as less than 35 nucleic acids, for example less than 30 nucleic acids, such as less than 25 nucleic acids, for example less than 20 nucleic acids, such as less than 15 nucleic acids, for example less than 10 nucleic acids, such as less than 8 nucleic acids, for example less than 6 nucleic acids.

3. Nucleic acid (Antimir) for use according to claim 1, wherein the nucleic acid is selected from the group consisting of SEQ ID NOS 1-11.

4. Nucleic acid (Antimir) for use according to claim 1, wherein the bone loss is associated with ankylosing spondylitis, renal osteodystrophy, osteoporosis, glucocorticoid-induced osteoporosis, Paget's disease, abnormally increased bone turnover, periodontitis, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), or bone loss associated with renal tubular disorders, or bone loss associated with bed-ridden conditions.

5. Nucleic acid (Antimir) for use according to claim 1, wherein the bone loss is associated with osteoporosis.

6. A method for reducing the levels of endogenous mir-138 in a cell, said method comprising the introduction of the nucleic acid and/or the vector according to the present invention into a cell in an amount sufficient to reduce the levels of said mir-138.

7. The method of claim 6, wherein the one or more nucleic acids are selected from the group consisting of SEQ ID NOS 1-11.

8. A method for treating or preventing bone loss in a patient comprising the steps of providing one or more nucleic acids (Antimir) capable of hybridizing to a mir-138, and administering said one or more nucleic acids to the patient.

9. The method according to claim 8, wherein said one or more nucleic acids comprises less than 50 nucleic acids, such as less than 45 nucleic acids, for example less than 40 nucleic acids, such as less than 35 nucleic acids, for example less than 30 nucleic acids, such as less than 25 nucleic acids, for example less than 20 nucleic acids, such as less than 15 nucleic acids, for example less than 10 nucleic acids, such as less than 8 nucleic acids, for example less than 6 nucleic acids.

10. The method of claim 8, wherein the one or more nucleic acids are selected from the group consisting of SEQ ID NOS 1-11

11. The method of claim 8, wherein the bone loss is associated with ankylosing spondylitis, renal osteodystrophy, osteoporosis, glucocorticoid-induced osteoporosis, Paget's disease, abnormally increased bone turnover, periodontitis, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), or bone loss associated with renal tubular disorders, or bone loss associated with bed-ridden conditions.

12. The method of claim 8, wherein the bone loss is associated with osteoporosis.
